(19) United States
(12) Patent Application Publication
(10) Pub. No.: US 2002/0124280 A1
(43) Pub. Date: Sep. 5, 2002

(54) Methods for the controlled, automatic excision of heterologous DNA from transgenic plants and DNA-excising gene cassettes for use therein

(76) Inventors: Yi Li, Mansfield Center, CT (US); Colum P. O'Donnell, Carlisle, MA (US); Hui Duan, Storrs, CT (US); Yan Hong Wu, Mansfield Center, CT (US); Richard McAvoy, Mansfield Center, CT (US)

Correspondence Address: Cummings & Lockwood, P.O. Box 1960, New Haven, CT, 06506-1960, US

(21) Appl. No.: 09/916,780

(22) Filed: Jul. 27, 2001

Related U.S. Application Data
(63) Continuation of application No. 60/221,318, filed on Jul. 28, 2000.

(51) Int. Cl. A01H 5/00

(52) U.S. Cl. 800/278; 800/282

(57) ABSTRACT

As disclosed herein, the present invention is directed to unique gene cassettes, and methods for their use, wherein the gene cassettes comprise multifunctional transgenic DNA sequences that completely, or nearly completely, excise themselves from the genome of plants into which they are introduced. The excision process is triggered in response to specific internal or external stimuli by means of excision/recombinase systems in unique combinations and orientations within the multifunctional transgenic sequences. Complete, or nearly complete, removal of the heterologous DNA significantly reduces the possibility of uncontrolled propagation of the transgenic species and may, more importantly, permit crops produced from transgenic plants to be co-mingled with non-transgenic crops for marketing purposes.

### REFERENCE TO RELATED APPLICATIONS

This application claims benefit under Title 35, U.S.C. §119(e), of U.S. application Ser. No. 60/221,318, filed Jul. 28, 2000.

### FIELD OF THE INVENTION

The present invention relates to genetically engineered DNA constructs, also referred to as gene cassettes, designed to automatically remove heterologous DNA sequences (transgenes) from the genome of transgenic plants after the transgenes have served their useful function and/or before their presence may give rise to concerns on behalf of the consuming public, or to a potentially negative impact of these transgenic species on the environment.

### BACKGROUND OF THE INVENTION

In particular, the present invention is directed to multifunctional transgenic sequences that completely, or nearly completely, excise themselves from the genome of transgenic plants in response to specific internal or external stimuli by means of excision/recombinase systems in unique combinations and orientations within the multifunctional transgenic sequences. Complete, or nearly complete, removal of the heterologous DNA significantly reduces the possibility of uncontrolled propagation of the transgenic species and may, more importantly, permit crops produced from transgenic plants to be co-mingled with non-transgenic crops for marketing purposes. Furthermore, the practice of the present invention provides a mechanism through which the creators of the transgenic species may gain commercial benefit from the marketplace dissemination of transgenic plants without concern for dissipation of the value of the proprietary technology contained therein. At the same time, and of critical importance for areas of the world with less than adequate supplies of plant-derived food stuffs, the methods of the present invention result in a wild-type plant species, after removal of the heterologous DNA, that is fully capable of further propagation.

The International Service for the Acquisition of Agri-Biotech Applications (ISAAA) estimates that global sales of transgenic crops were $2.1 to $2.3 billion in 1999. A total of 39.9 million hectares of transgenic crops were planted throughout the world in 1999, of which herbicide tolerant crops (primarily “Roundup Ready” technology) represented 28.1 million (71%) hectares, and B.t. insect resistance corn lines represented 8.9 million hectares (22%). It is estimated that farmers throughout the world paid a total of $600 million in 1999 for the right to plant transgenic crops. ISAAA projects that global sales of transgenic crops will grow to $8 billion in 2005 and $25 billion in 2010.

According to the US Census Bureau, world population is expected to grow by 55% from almost 6 billion people in 1999 to 9.3 billion in 2050. The increased demand for food and fiber combined with a reduction in available agricultural lands makes it imperative that agricultural production efficiency increases dramatically as the population does. Quantity and quality improvements resulting from the technology that contributed to the first “green revolution,” including conventional Mendelian plant breeding methods, the application of chemical fertilizers and pesticides, and increased mechanization of agriculture, have reached their practical limits. Some of these same tools now contribute to environmental problems through chemical contamination and soil erosion.

The first transgenic plants were commercialized in 1995 and have since been proven to be the most successful new-products in agricultural history. In addition to the economic benefits of the technology, the environmental benefits are also potentially significant. For example, in the first several years of the use of transgenic crops transformed with the Bat. insecticide gene, the use of chemical insecticides decreased significantly. Herbicide tolerant crops such as “Roundup Ready” corn have allowed the expansion of no-till farming which significantly reduces the extent of soil erosion. It is expected that over the next fifteen to thirty years, agriculture, horticulture, and forestry will be transformed by a second “green revolution” resulting from genetic engineering of plants. Transgenic plant technology will contribute to increased production efficiency of agriculture, reductions in pesticide, growth regulator and fertilizer pollution, improvements in food quality and nutritional value, and extended shelf life of both food and horticultural crops, development of new ornamental crops, and production of more cost-effective industrial enzymes and pharmaceuticals.

Such plants, however, have recently been subject to increased scrutiny from both the public and local and national governments due to a perception that such genetically engineered species pose safety and health risks. Among the issues surrounding the development and widespread use of genetically-modified (GM) plants are the following:

some enzymes and other types of proteins are allergens, and plant species genetically modified to express these proteins can cause allergic reactions in some people;

when the antibiotic resistance genes commonly used as selection markers in genetic engineering technology remain in food crops, consumption over time may lead to an acquired resistance to the effectiveness of similar pharmaceutical antibiotics used to treat infections in humans;

transgenic crops that are planted near non-transgenic counterparts may cross-pollinate causing the progeny of the non-transgenic relative to be transgenic which raises the concern of uncontrolled propagation of transgenic plant species and the heterologous genes contained therein.

the results of recent studies have been interpreted to indicate that pollen from corn varieties genetically engineered to contain a bacterial gene that is toxic to certain species of lepidopteran insects (B.t. corn) was responsible for the destruction of Monarch butterflies; however, more recent research indicates that there may be no harm to Monarch butterflies from the most widely used forms of B.t. corn (Obrycki, J., et al., BioScience, 51: 353-361 (2001);

for crop species genetically modified to enhance resistance to certain herbicides, controlling second-generation re-growth and sucker shoots can be difficult, raising the specter of uncontrolled propagation of such herbicide-resistant crops to the point where they become weedy pests.

From an historical perspective, the agricultural and biotechnology industries are facing a watershed of progress not unlike that faced by the agricultural industry in the latter stages of the nineteenth century. As the agronomy-based economy and society of the eighteenth and nineteenth centuries evolved into the industrial economy of the twentieth century, difficult choices were presented to government and society. Less and less of the available acreage of the industrially developed nations of the world was dedicated to the production of food crops. At the same time, world population started an explosion that has yet to abate. In light of this, the agricultural industry was forced to rely increasingly on the use of chemicals such as fertilizers, insecticides and herbicides to achieve crop production levels sufficient to feed growing populations. The consequences of the environmental dilemmas that such reliance engendered has been well documented in the twentieth century. It is undisputed that indiscriminate usage of agricultural chemicals has had significant environmental consequences, and will continue to do so. However, it is (or should be) equally undisputed that the enhanced yields and agricultural productivity made possible through technological advances throughout the last century have prevented the starvation of significant segments of the world's population. Similar dilemmas once again face the agricultural industry as technology, including the genetic engineering of crop species, makes it possible to achieve highly desirable phenotypic traits previously unattainable through plant development programs based solely on traditional Mendelian genetics.

In recent years, a number of approaches to the control of plant gene expression have been developed that provide tools that may, if applied properly, have some utility in addressing issues pertaining to genetically modified plants. For example, the Delta and Pine Land Company, in an attempt to protect the proprietary content of genetically modified crops, has developed technology for the control of plant gene expression that has become known as “terminator” technology. This technology has been disclosed in U.S. Pat. Nos. 5,723,765, 5,925,808 and 5,977,441, the disclosures of which are specifically incorporated herein by reference. In general, these patents disclose methods for excising DNA sequences associated with transgenes from genetically modified plants based on the use of recombinase/excision systems. According to this technology, a transgene of interest, usually a gene conferring a desirable phenotypic trait on the transformed plant, is operatively coupled to a lethal gene so that, when the recombinase/excision system is triggered, a gene is also activated that renders the resulting plants incapable of further propagation. In other words, using genetically modified corn as an example, the plant is transformed with a gene conferring a desirable phenotypic trait such as insect or herbicide resistance. As the plant matures, the plant displays the desired trait. Thus, a farmer will be able to produce a crop from a planting of seeds for the transgenic species. However, due to the linking of the desired transgene to lethal gene, the seeds produced from this transformed plant cannot be planted to grow additional crops with the transgenic trait. Thus, the transgenic crop will have value for the farmer only for its nutritional or food content, and not a source of seed for further plantings. From a business perspective, the genetically engineered obsolescence of the crop makes considerable economic sense because it guarantees a market for the next season's seeds. This technology also serves as a built-in protection for the developer against the uncontrolled exploitation of the proprietary content of the genetically modified plant line. However, it does nothing for farmers in less developed areas of the who, because of economic necessity, must rely on a portion of one season's crop as the source of seeds for the next season's plantings. Almost as an afterthought, this technology does provide some benefit in light of the issues discussed above in that it insures that it will be virtually impossible for transgenes contained within the plants to propagate in an uncontrolled manner.

According to the disclosures of these patents, the functioning of the recombinase/excision system is under control of an externally-inducible promoter system. Upon activation of the promoter in response to an external signal, the recombinase system effectively excises a sequence flanked by the excision sites. Because the excised sequence has acted as a blocking sequence for one or more transgenic sequences, or has expressed a repressor for the transgene, the removal of the blocking sequence permits the active expression of the previously dormant transgene. However, the transgene is also linked to a lethal gene so that when the desired gene is activated, functioning of the lethal gene assures that the next generation of the transformed plant is incapable of germination and successive proliferation.

This approach is specifically designed for implementation with hybrid seed lines where the individual parent lines can contribute separate segments of the system. Such inbred parents will not display the altered phenotype, and will produce seed that would give rise to plants that also do not display the altered phenotype. When the external stimulus to which the repressor is sensitive is applied to this seed or this plant, the repressor no longer functions, permitting the expression of the site-specific recombinase, or alternatively, when the recombinase is introduced via hybridization it is expressed during germination of the seed, either of which effects the removal of the blocking sequence between the specific excision signal sequences. Upon removal of the blocking sequence, the transiently-active promoter becomes directly linked to the gene whose expression results in an altered plant phenotype. A plant grown from either treated or hybrid seed, or a treated plant, will still not exhibit the altered phenotype until the transiently-active promoter becomes active during the plant's development, after which the gene to which it is linked is expressed, and the plant will exhibit an altered phenotype.

If the transiently-active promoter is one that is active only in late embryogenesis, the gene to which it is linked will be expressed only in the last stages of seed development or maturation. If the gene linked to this promoter is a lethal gene, it will render the seed produced by the plants incapable of germination. In the initially-transformed plant cells, this lethal gene is not expressed, not only because the promoter is intrinsically inactive, but because of the blocking sequence separating the lethal gene from its promoter. The repressor is expressed constitutively and represses the expression of the recombinase. These plant cells can be regenerated into a whole plant and allowed to produce seed. The mature seed is exposed to a stimulus, such as a chemical agent, that inhibits the function of the repressor. Upon inhibition of the repressor, the promoter driving the recombinase gene is depressed and the recombinase gene is expressed. The resulting recombinase recognizes the specific excision sequences flanking the blocking sequence, and effects the removal of the blocking sequence. The late embryogenesis promoter and the lethal gene are then directly linked. The lethal gene is not expressed, however, because the promoter is not active at this time in the plant's life cycle. This seed can be planted, and grown to produce a desired crop of plants. As the crop matures and produces a second generation of seed, the late embryogenesis promoter becomes active, the lethal gene is expressed in the maturing second generation seed, which is rendered incapable of germination. In this way, accidental reseeding, escape of the crop plant to areas outside the area of cultivation, or germination of stored seed can be avoided.

Other approaches making use of recombinase/excision systems, have been reported. For example, in a series of patents assigned to Purdue University, recombinase/excision systems have been disclosed that are capable of site specific transformation accompanied by removal of unwanted or misplaced copies of the transgene of interest. Included among these are U.S. Pat. Nos. 5,527,695, 5,744,336, 5,910,415, and 6,110,736, the disclosures of which are incorporated herein by specific reference. The main thrust of the disclosures of these patents is the transformation of plants with transgenes imparting desirable phenotypic traits targeted to specific sites within the plant's genome, accompanied by removal of extra or misplaced copies of the gene of interest. This is accomplished, according to a method termed homologous recombination, by transformation of the plant with a transgene sequence that comprises DNA sequences homologous to the targeted sites. The transgene sequences also comprise recombinase/excision systems the inclusion of which are designed to result in the removal of random copies of the transgene of interest, including those that are inserted into the plant's genome at other than the desired, targeted sites. According to the disclosures of these patents, an homologous recombination event results in less than the full transgene sequence being inserted into a targeted site, whereas a random recombination event results in complete insertion. The difference between the sequences transformed into the plant is the presence of the excision sites for a specific recombinase. Thus, in theory, the random insertions are excised upon expression of the recombinase gene. However, the remaining transgene sequences, which may include selectable marker sequences such as those conferring antibiotic resistance, remain in the transformed plant's genome and are propagated in succeeding generations.

Other approaches, such as those disclosed in U.S. Pat. Nos. 4,959,317 and 5,658,772, the disclosures of which are incorporated herein by specific reference, involve the use of recombinase/excision systems in the site-specific transformation of hybrid plant species. According to these disclosures, a parental inbred line is transformed with a gene designed to render the parent male sterile. This inbred is then used as the female parent for creation of a hybrid species. However, due to the fact that the male sterile plant is heterozygous for the sterility gene that acts as a dominant trait, only 50% of the plants grown from the hybrid seed are fertile. Introduction of components of a recombinase/excision system through the other inbred parent line allows for the restoration of male function in the plant with a resulting higher level of fertility in the next generation. In general, these patents disclose a system whereby components of a recombinase/excision system are introduced into a hybrid separately through parental inbred lines. In the resulting hybrid, the recombinase/excision system functions to remove a DNA sequence that blocks or represses a sequence imparting a desirable phenotypic trait to the plant line. However, this approach does little to address the concerns raised by further propagation of the transgenic phenotype, along with associated selectable marker genes, in succeeding generations of hybrid.

In addition, other approaches have been reported that are designed to address some of the problems associated with GM plants discussed above. For example, Dale and Ow, in “Gene transfer with subsequent removal of the selection gene from the host genome,” Proc. Natl. Acad. Sci. USA, 88: 10558-10562 (1991), have disclosed an approach based on the use of recombinase/excision systems to remove the selectable marker genes (such as those conferring antibiotic resistance) after selection of successfully transformed plants. In general, the authors addressed a specific problem associated with selection of transforms in successive, step-wise transformation processes due to the lack of availability of suitable selection markers. In such a process, the presence of the selectable marker gene in transformed plants would make it impossible to select successfully transformed plants after successive transformation events. This is overcome by removal of the marker gene, via a recombinase/excision system under the control of a transiently active promoter, following selection and before the subsequent transformation steps. However, the resulting transformed plants retain the transgene imparting a desired phenotypic trait with which the marker gene is associated. The authors also recognized the unintended positive consequence of the removal of antibiotic resistance genes due to concerns over continued exposure to such genes and their potential to lead to resistance to antibiotics used on humans. However, as would be fully appreciated by one of skill in the appropriate art, it is a relatively simple process to control the expression of a recombinase to remove only a marker gene following the selection process. It is a far more complex problem to precisely control the operation of a recombinase/excision system so as to remove essentially all transgenes and their expression products only after the transformed plant displays the desired trait. It is even more difficult to do so in a manner that restores both the wild-type genome and the ability for further propagation of the transformed species.

Consequently, there remains a need for a system by which a transgene of interest can be introduced into a plant in order to confer a desirable phenotypic trait and, after display of that trait by the transformed plant, restore the plant to its fertile, wild-type state.

### SUMMARY OF THE INVENTION

In a first embodiment, the present invention provides a method for the creation of a transiently transgenic plant whereby a heterologous transgene temporarily conveys a desirable phenotypic trait to the plant, the method comprising the steps of (a) constructing a gene cassette comprising (i.) one or more DNA sequences for a gene conferring a desirable phenotypic trait; (ii.) one or more DNA sequences expressing a recombinase-type protein; (iii.) at least one pair of DNA excision sequences cleavable by the recombinase-type protein, wherein the excision sequences flank the heterologous DNA; and (iv.) a transiently activated promoter operably linked to the DNA sequence expressing the recombinase-type protein and controlling expression of the recombinase-type protein, wherein the promoter is activated, and thereby directs expression of the recombinase-type protein, in response to developmental or external stimuli; (b) introducing the cassette into the genome of the plant; and (c) exposing the DNA sequences within the cassette to a stimulus that activates the promoter, whereby the promoter directs expression of the recombinase-type protein, and the recombinase-type protein excises the heterologous DNA from the genome of the plant.

Preferably, the gene cassette further comprises a DNA sequence for a marker gene. Also preferably, the transiently-active promoter is activated only in certain organs of the plant, only at specific stages in the plant's developmental cycle, or alternatively, in response to an external stimulus. An external stimulus may comprise, for example, exposure to a specific chemical species. Without limitation, and as would be recognized by one of ordinary skill in the appropriate art, the chemical stimulus may comprise exposure to ethanol, ecdysone, a glucocorticoid such as dexamethasone (DEX), antobiotics such as tetracycline, forms of estrogen such as estradiol, or heavy metals such as cadmium (Cd²⁺) and copper (Cu²⁺). Alternatively, the transiently-activated promoter of the method of the present invention may be activated in response to, or in combination with, other external stimuli such as heat shock, exposure to electromagnetic radiation, or exposure to reduced temperatures. In addition, the transiently-activated promoters may be activated in specific tissues of a plant or at certain specific periods in a plant's developmental cycle only when exposed to an external stimulus, such as those enumerated immediately above.

In another embodiment, the present invention provides a gene cassette for the reversible introduction of heterologous DNA sequences into a genome of a vegetatively propagated plant, the gene cassette comprising (a) a DNA sequence for one or more genes that express a recombinase-type protein; (b) a DNA sequence for one or more transiently-active promoters operably linked to the one or more DNA sequences; (c) one or more pairs of DNA excision sequences, wherein the excision sequences are each cleavable by the recombinase-type protein; and (d) one or more heterologous DNA sequences capable of conferring a desirable phenotypic trait into the plant into which the cassette is introduced, wherein the one or more heterologous sequences are flanked by at least one of the pairs of excision sequences. Preferably, the one or more pairs of excision sequences are recognized only by the recombinase-type protein expressed by the recombinase gene of the cassette. Also, this embodiment of the present invention contemplates that the cassette may further comprise one or more selectable marker DNA sequences, such as the gene conferring resistance to the antibiotic kanamycin.

In this embodiment, the disclosed invention provides that the promoter sequence is activated only in response to internal stimuli or signals such as those provided in certain organs of the plant, or associated with specific stages in the plant's developmental cycle. Alternatively, the transiently-activated promoters of the gene cassette of the present invention may be activated in response to an external or environmental stimulus. Such an external stimulus may comprise, for example, exposure to a specific chemical species. Without limitation, and as would be recognized by one of skill in the appropriate art, the chemical stimulus may comprise exposure to ethanol, ecdysone, a glucocorticoid such as dexamethasone (DEX), antobiotics such as tetracycline, forms of estrogen such as estradiol, or heavy metals such as cadmium (Cd²⁺) and copper (Cu²⁺). In addition, the transiently-activated promoter of the gene cassette of the present invention may be activated in response to, or in combination with, other external stimuli such as heat shock, exposure to electromagnetic radiation, or exposure to reduced temperatures. The present invention also provides a gene cassette where activation of the selectively-activated promoter is achieved only in response to external stimuli, and even then only in certain organs or tissues of the plant, some of which may exist only at specific stages in the plant's developmental cycle.

Preferably, the gene sequence of the cassette expresses a protein of the type selected from the group consisting of recombinases, invertases, integrases, transposases and resolvases. More preferably, the sequence expresses a recombinase-type protein selected from the group consisting of FLP, Cre, R, Gin, PIV, C31, FimB, KW, SSV, IS1110/IS492, ParA, and TnpX.

Furthermore, the gene cassette of the present invention comprises one or more promoter gene sequences selected from the group consisting of organ- or developmental stage-specific gene promoters selected from the group consisting of seed-, fruit-, pollen-, stem/shoot-, leaf-, root-specific gene promoters. Preferably, the one or more promoter gene sequences are selected from the group consisting of AG (SEQ. ID NO. 7), AGL5 (SEQ. ID NO. 6), Bcp1 (SEQ. ID NO. 5), LAT52 (SEQ. ID NO. 8), PLENA, avrRpt2, and alc.

In an alternative embodiment, the present invention provides a gene cassette for the reversible introduction of heterologous DNA sequences into a genome of a sexually propagated plant, the gene cassette comprising (a) a first DNA sequence comprising (i.) a sequence that expresses a first recombinase-type protein; (ii.) a sequence for an inducible promoter, operably linked to the sequence that expresses the first recombinase-type protein, wherein the promoter is capable of being activated in reaction to an external stimulus; (iii.) a sequence that expresses a transcription factor capable of regulating transcriptional activity of the sequence that expresses the first recombinase-type protein under control of the sequence for the externally inducible promoter; and (iv.) one or more pairs of DNA excision site sequences wherein the excision sites are capable of being cleaved only by the first recombinase-type protein; (b) a second DNA sequence comprising (i.) a sequence capable of expressing a second recombinase-type protein; (ii.) a sequence for a transiently-active promoter capable of controlling expression of the second recombinase-type protein; and (iii.) one or more heterologous DNA sequences capable of conferring a desirable phenotypic trait on the genome into which the cassette is introduced; and (c) one or more pairs of DNA excision site sequences wherein the excision sites are capable of being cleaved only by the second recombinase-type protein.

This embodiment of the present invention further contemplates that the first DNA sequence is capable of blocking the induction of expression of the second recombinase-type protein by the transiently-active promoter. The gene cassette of the present embodiment further contemplates that the second DNA sequence comprises (a) a sequence capable of expressing a third recombinase-type protein; (b) a sequence capable of expressing a protease protein; and (c) one or more pairs of DNA excision site sequences wherein the excision sites are capable of being cleaved only by the third recombinase-type protein, wherein the third recombinase-type protein is linked to the second recombinase-type protein through a specific amino acid sequence capable of being cleaved by the protease protein. Preferably, the protease protein is NIa (nuclear inclusion protein protease). The gene cassette also provides that the second DNA sequence further comprises a second promoter sequence operatively linked to the sequence that expresses the protease protein. Furthermore, the amino acid sequence linking the second recombinase-type protein to the third recombinase-type protein comprises the following amino acid sequence: V-R-T-Q-G-P-K-R.

This embodiment provides that the second DNA sequence further comprises a marker gene sequence that, preferably, confers resistance to an antibiotic such as kanamycin. It is also preferred that the transiently-active promoter sequence is activated only in certain organs of the plant or, alternatively, only at specific stages in the plant's developmental cycle. As a further alternative, the externally-inducible promoter sequence is activated in response to an external or environmental stimulus. Such an external stimulus may comprise, for example, exposure to a specific chemical species. Without limitation, and as would be recognized by one of skill in the appropriate art, the chemical stimulus may comprise exposure to ethanol, ecdysone, a glucocorticoid such as dexamethasone (DEX), antobiotics such as tetracycline, forms of estrogen such as estradiol, or heavy metals such as cadmium (Cd²⁺) and copper (Cu²⁺).

In addition, the transiently-activated promoter of the gene cassette of the present invention may be activated in response to, or in combination with, other external stimuli such as osmotic stress, heat shock, exposure to electromagnetic radiation, or exposure to reduced temperatures. The present invention also provides a gene cassette where activation of the selectively-activated promoter is achieved only in response to external stimuli, and even then only in certain organs or tissues of the plant, some of which may exist only at specific stages in the plant's developmental cycle.

Preferably, the gene sequence of the cassette of this embodiment of the present invention expresses a protein of the type selected from the group consisting of recombinases, invertases, integrases, transposases and resolvases. More preferably, the sequence expresses a recombinase-type protein selected from the group consisting of FLP, Cre, R, Gin, PIV, C31, FimB, KW, SSV, IS 1110/IS492, ParA, and TnpX.

Furthermore, the gene cassette of the present invention comprises one or more promoter gene sequences selected from the group consisting of organ- or developmental stage-specific gene promoters selected from the group consisting of seed-, fruit-, pollen-, stem/shoot-, leaf-, root-specific gene promoters. Preferably, the one or more promoter gene sequences are selected from the group consisting of AG, AGL5, Bcp1, LAT52, PLENA, SIM, avrRpt2, and alc.

### BRIEF DESCRIPTION OF THE DRAWINGS

FIG. 1, in two panels, FIGS. 1A and 1B, provides a schematic representation of a gene cassette of the present invention:

FIG. 1A is a schematic representation of the various elements comprising a gene cassette for use with vegetatively propagated plants;

FIG. 1B is a schematic representation of the gene cassette described in Example 1.

FIG. 2, in two panels, FIGS. 2A and 2B, provides a schematic representation of a gene cassette of the present invention:

FIG. 2A is a schematic representation of the various elements comprising a gene cassette for use with vegetatively propagated plants;

FIG. 2B is a schematic representation of the gene cassette described in Example 2.

FIG. 3, in two panels, FIGS. 3A and 3B, provides a schematic representation of a gene cassette of the present invention:

FIG. 3A is a schematic representation of the various elements comprising a gene cassette for use with sexually propagated plants;

FIG. 3B is a schematic representation of the gene cassette described in Example 3.

### DETAILED DESCRIPTION OF THE INVENTION

To date, as discussed above, a number of procedures have been disclosed for excision of a portion of transgene sequences from the genome of a transformed plant using DNA recombinase systems. However, it is technically much more challenging to excise essentially all transgenes from a transformed plant, including recombinase genes driving the excision process, only at the stage and generation when the functions of the transgenes are no longer needed. Using DNA recombination systems from microorganisms, we have developed gene cassettes for the controlled, automatic excision of all transgenes from transformed plants, either in specific organs (e.g., seeds, fruits and pollen), or at specific stages in the plant's developmental cycle. This excision occurs only after the transgenic functions are either no longer needed, or the continued presence of transgenes could cause concern. By doing so, potential negative effects of heterologous gene sequences can be significantly reduced or eliminated. In some cases, the excision of transgenes is under the direct control of an organ and/or developmental stage gene promoter. In other cases, a cascade of events triggered by an external stimulus such as exposure to a chemical leads to excision of the heterologous gene material. To that end, plant-active gene promoter sequences have been carefully chosen to control expression of DNA recombinase sequences. Also, two or more different DNA recombination systems have been engineered into a single cassette construct to ensure complete or near complete excision of transgenes, and to rearrange the targeted DNA sequences within the gene cassette at a predetermined time so that temporal and spatial control of the activation of the excision mechanism is achieved.

Therefore, in a first embodiment, the present invention provides a method for the creation of a transiently transgenic plant whereby a heterologous transgene temporarily conveys a desirable phenotypic trait to the plant, the method comprising the steps of (a) constructing a gene cassette comprising (i.) one or more DNA sequences for a gene conferring a desirable phenotypic trait; (ii.) one or more DNA sequences expressing a recombinase-type protein; (iii.) at least one pair of DNA excision sequences cleavable by the recombinase-type protein, wherein the excision sequences flank the heterologous DNA; and (iv.) a transiently activated promoter operably linked to the DNA sequence expressing the recombinase-type protein and controlling expression of the recombinase-type protein, wherein the promoter is activated, and thereby directs expression of the recombinase-type protein, in response to developmental or external stimuli; (b) introducing the cassette into the genome of the plant; and (c) exposing the DNA sequences within the cassette to a stimulus that activates the promoter, whereby the promoter directs expression of the recombinase-type protein, and the recombinase-type protein excises the heterologous DNA from the genome of the plant.

The creation of a transformed cell requires that exogenous DNA be physically placed within the host cell. Current transformation procedures utilize a variety of techniques to introduce DNA into a cell. In one form of transformation, the DNA is microinjected directly into cells through the use of micropipettes. Alternatively, high velocity ballistics can be used to propel small DNA associated particles into the cell. In another form, the cell is permeablized by the presence of polyethylene glycol, thus allowing DNA to enter the cell through diffusion. DNA can also be introduced into a cell by fusing protoplasts with other entities which contain DNA. These entities include minicells, cells, lysosomes or other fusible lipid-surfaced bodies. Electroporation is also an accepted method for introducing DNA into a cell. In this technique, cells are subject to electrical impulses of high field strength that reversibly permeablize biomembranes, allowing the entry of exogenous DNA sequences.

In addition to these “direct” transformation techniques, transformation can be performed via bacterial infection using Agrobacterium tumefaciens or Agrobacterium rhizogenes. These bacterial strains contain a plasmid (referred to as Ti or Ri, respectively) that is transmitted into plant cells after infection by Agrobacterium. One portion of the plasmid, named transferred DNA (T-DNA), is then integrated into the genomic DNA of the plant cell. Agrobacterium-mediated transformation works best with dicotyledonous diploid plant cells whereas the direct transformation techniques work with virtually any cell. Direct transformation techniques can also be used to transform haploid cells obtained from immature inflorescences of plants. As would be recognized by one of ordinary skill in the relevant art, these techniques have been extensively described in the literature and can be adapted to introduce foreign genes and other DNA sequences into plant cells. As technology for genetic manipulation continues to develop, transformation events that formerly required a considerable level of skill, experience and training to accomplish on a predictable basis have become nearly routine. Indeed, the laboratory manipulations necessary to achieve such transformations, although once the basis of Nobel prize level research, are now the subject of undergraduate biology teaching laboratory exercises. One of ordinary skill in the art may consult, guided by the disclosure contained herein, routinely available reference sources for all of the procedural detail necessary to accomplish the cassette construction and transformations needed in the practice of the present invention. For example, reference may be had to Maniatis et al., Molecular Cloning: A Laboratory Manual, Cold Springs Harbor Laboratory, New York: 1982, for details of culturing bacteria, preparing and manipulating DNA sequences. Materials needed for these procedures, such as reagents, buffer solutions, culture media, restriction endonucleases, and the like are readily available from a number of commercial sources such as New England Biolabs, Inc., of Beverly, Mass. [http://www.neb.com/], Roche Diagnostics [http://www.roche.com/diagnostics/], or other major commercial suppliers accessible via conventional marketing channels, including the Internet.

Of the available plant transformation techniques well-known to workers in the art, any technique that is suitable for the target plant species can be employed in the practice of the present invention. For example, the sequences can be introduced in a variety of forms, such as a strand of DNA, in a plasmid, or in an artificial chromosome, to name a few. The introduction of the sequences into the target plant cells can be accomplished by a variety of techniques, such as those described above. Those of ordinary skill in the art can refer to literature sources such as those mentioned above for details, and select suitable techniques and materials without undue experimentation.

A gene that results in an altered plant phenotype is any gene whose expression leads to the plant exhibiting a trait or traits that would distinguish it from a plant of the same species not expressing the gene. Examples of such altered phenotypes include a different growth habit, altered flower or fruit color or quality, premature or late flowering, increased or decreased yield, sterility, mortality, disease susceptibility, altered production of secondary metabolites, or an altered crop quality such as taste or appearance. In the cassette used in the practice of the present invention, a gene and a promoter are considered to be operably linked if they are on the same strand of DNA, in the same orientation, and are located relative to one another such that the promoter directs transcription of the gene. The presence of intervening DNA sequences between the promoter and the gene does not preclude an operable relationship.

Essential to the functioning of the cassettes of the present invention are site-specific recombinase systems. Such systems generally comprise three separate elements: two pairs of DNA sequences (the site-specific recombination sequences) and a specific enzyme (the site-specific recombinase). The site-specific recombinase will catalyze a recombination reaction only between two site-specific recombination sequences. The recombinase/excision sequence systems used in the practice of the present invention can be any one that selectively removes DNA in a plant genome under appropriate control. The target excision sequences are preferably unique in the plant, so that unintended cleavage of the plant genome does not occur. Several examples of such systems are discussed in U.S. Pat. No. 4,959,317, discussed above. As would be appreciated by one of ordinary skill in the appropriate art, other recombinase/excision systems would have utility in the practice of the present invention.

For illustrative purposes, and without limit to the scope of practice of the claimed invention, such systems include the Cre/loxP system (“Cre”—causes recombination; “loxP”—locus of crossing over) of bacteriophage P1, the FLP/FRT system (“FRT”—FLP recognition target) of yeast, the Gin recombinase of phage Mu, the Pin recombinase of E. coli, and the R/RS system of the pSR1 plasmid. The two preferred site specific recombinase systems are the bacteriophage P1 Cre/lox and the yeast FLP/FRT systems. For example, the cre gene (SEQ. ID NO. 2) encodes the Cre recombinase that recognizes its site-specific recombination sequence, loxP (SEQ. ID NO, 1) to invert or excise the intervening sequences. Similarly, the flp gene (SEQ. ID NO. 4) encodes the FLP recombinase that recognizes its own site-specific recombination sequence, FRT (SEQ. ID NO. 3). The recognition sequences for each of these two systems are relatively short (34 base pairs (bp) for loxP (SEQ. ID NO. 1) and 47 bp for FRT (SEQ. ID NO. 3).

By way of illustration, the 34-bp loxP site, consisting of two 13-bp inverted repeats separated by an 8-bp asymmetric spacer region, is recognized by the 38 kDa Cre recombinase protein. These two elements interact to affect DNA recombination in vitro, resulting in excision, inversion, or insertion of DNA depending on the location and orientation of the loxP sites. Since the loxP site is an asymmetrical nucleotide sequence, two loxP sites on the same DNA molecule can have the same or opposite orientations with respect to each other. Recombinations between lox sites in the same orientation result in deletion of the DNA segment located between the two lox sites and a connection between the resulting ends of the original DNA molecule. As much as 200 kilobases can be removed between two loxP sites. The original DNA molecule and the resulting circular molecule comprising the excised sequence each contain a single loxP site. Recombination between lox sites in opposite orientations on the same DNA molecule result in an inversion of the nucleotide sequence of the DNA segment located between the two loxP sites. In addition, reciprocal exchange of DNA segments proximate to loxP sites located on two different DNA molecules can occur. All of these recombination events are catalyzed by the 38 kDa Cre protein.

The FLP/FRT system of yeast is also particularly preferred because it normally functions in a eukaryotic organism (yeast), and is well characterized. It is believed that the eukaryotic origin of the FLP/FRT system allows the FLP/FRT system to function more efficiently in eukaryotic cells than the prokaryotic site-specific recombinase systems.

The RS/R system is another site-specific recombination system from Z. rouxii. Similar to Cre and FLP, the r gene (SEQ. ID NO. 10) encodes the R protease that catalyzes excision of the DNA sequences that are flanked by two RS sites (SEQ. ID NO. 9). The RS/R system has been shown to be effective to remove antibiotic resistance genes from transgenic plants or delete blocking DNA sequences within functional genes in higher plants.

According to the practice of the present invention, essentially all heterologous DNA sequences can be excised from the genomes of the transformed plants and their products (mRNA and proteins), present in specific organs at a specific developmental stage. Based on knowledge of the average life span of RNA's and proteins in the cell, it is expected that transgene products should be completely depleted several days after deletion of the transgenes from the host genome. Thus, if transgenes are excised from the host genome in response to an appropriate stimulus several days prior to harvesting or marketing, potential negative effects of the transgenes can be reduced or eliminated. This technology will lead to a fully functional nontransgenic (or wild-type) yield from transgenic plants. The progeny of such plants will also be essentially free of the transgene sequences originally present in the preceding generation of plant. This technology will help to reduce potential health implications of transgenic food, to eliminate undesirable spread of transgenes to the environment because pollen and seeds produced from transgenic plants are non-transgenic, and to protect proprietary rights inherent in the transgenic technology.

With some minor modifications of the gene cassettes, the proposed technology can be used to remove transgenes from any specific organ or whole plant or at any desirable developmental stage once appropriate gene promoters are used to control the recombinase gene expression. For instance, if a potato tuber specific gene promoter is used, non-transgenic potato tubers can be produced from transgenic plants that are either resistant to insects or diseases or have improved photosynthetic activity. When a low temperature-inducible gene promoter is used, gene excision will occur when plants are at low temperatures, which can be useful for green vegetables and fresh fruits because they are often stored and transported at reduced temperatures. If the trait gene is for enhancement of concentrations of an essential amino acid in seeds, a mid-and late-stage active, seed-specific gene promoter should be used to control expression of the recombinase genes.

The gene excision cassettes of the present invention automatically excise heterologous DNA sequences from the genome of a host plant when the transgene's functions are no longer needed, or when their continued presence may give rise to concerns over potential side effects. As a consequence, practice of the present invention will make it possible to produce non-transgenic food products from transgenic plants; to potentially expand the current market for transgenic plants and crops by overcoming the ban in Europe on GM foods; to allow further penetration by transgenic species into appropriate markets around the world; to eliminate uncontrolled proliferation of transgenic species through out-crossing of wild-type counterparts; and to protect proprietary rights to inherent technology. Most importantly, all this can be attained while preserving the viability of seed produced by the genetically modified plants so that farmers can retain a portion of their seed crop for later re-planting. This is in direct contrast to the effects of use of “terminator” technology as developed by Delta and Pine Land Company, discussed above.

The economic potential of the present invention is significant because it can reduce or eliminate the negative environmental and health implications associated with GM plants, and should obviate the reasons for public concern over transgenic technology. It is anticipated that this will make possible not only continued use and expanded use of existing transgenics in the traditional agricultural industry, but also clear the way for a plethora of new applications in biochemical, fermentation and pharmaceutical industries because of low-cost productions of proteins and enzymes using transgenic plants as bioreactors.

Preferably, the gene cassettes used in the practice of the methods of the present invention further comprises a DNA sequence for a marker gene. Also preferably, the transiently-active promoter is activated only in certain organs of the plant, only at specific stages in the plant's developmental cycle, or alternatively, in response to an external stimulus. An external stimulus may comprise, for example, exposure to a specific chemical species. Without limitation, and as would be recognized by one of ordinary skill in the appropriate art, the chemical stimulus may comprise exposure to ethanol, ecdysone, a glucocorticoid such as dexamethasone (DEX), antobiotics such as tetracycline, forms of estrogen such as estradiol, or heavy metals such as cadmium (Cd²⁺) and copper (Cu²⁺). Alternatively, the transiently-activated promoter of the method of the present invention may be activated in response to, or in combination with, other external stimuli such as heat shock, exposure to electromagnetic radiation, or exposure to reduced temperatures. In addition, the transiently-activated promoters may be activated in specific tissues of a plant or at certain specific periods in a plant's developmental cycle only when exposed to an external stimulus, such as those enumerated immediately above.

Transformed cells (those containing heterologous DNA inserted into the host cell's DNA) can be selected from untransformed cells if a selectable marker was included as part of the introduced DNA sequences. Selectable markers include genes that provide antibiotic resistance or herbicide resistance. Cells containing these genes are capable of surviving in the presence of antibiotic or herbicide concentrations that kill untransformed cells. Examples of selectable markers include the bar gene which provides resistance to the herbicide Basta, the nptII gene which confers kanamycin resistance and the hpt gene which confers hygromycin resistance. The potentially undesirable effects of the inclusion of such marker genes in transformed plants is addressed at length above.

The controlled expression of transgenic DNA sequences is accomplished through the use of constructs comprising regulatory elements. Various gene expression control elements, or regulatory sequences, that are operable in one or more species of organisms are well known in the art. A regulatory sequence, in general, refers to a nucleotide sequence located proximate to a gene whose transcription is controlled by the regulatory nucleotide sequence in conjunction with the gene expression apparatus of the cell. The regulatory nucleotide sequence normally is located 5′ to the gene. The expression “nucleotide sequence” refers to a polymer of DNA or RNA, which can be single- or double-stranded, optionally containing synthetic, non-natural, or altered nucleotides capable of incorporation into DNA or RNA polymers. A regulatory sequence can include a promoter region, as that term is conventionally employed by those skilled in the art. A promoter region can include an association region recognized by an RNA polymerase, one or more regions which control the effectiveness of transcription initiation in response to physiological conditions, and a transcription sequence.

In general, regulatory elements can be operably linked to any gene to control the gene's expression, the entire unit being referred to as the “expression cassette.” An expression cassette will typically contain, in addition to the coding sequence, a promoter region, a translation initiation site and a translation termination sequence. Unique endonuclease restriction sites may also be included at the ends of an expression cassette to allow the cassette to be easily inserted or removed when creating DNA constructs. In the practice of the present invention, the gene cassettes contain specific excision sequences recognized solely by the recombinase proteins expressed by recombinase DNA sequences contained within the cassette in order to direct excision of the heterologous DNA within the cassette.

Although a preferred embodiment of the present invention contemplates the use of a gene cassette prepared from a single construct that is then transformed into the plant of choice, those of skill in the appropriate art will recognize that technology exists today that would permit the step-wise transformation of a target plant to effectively build in vivo a gene cassette of the present invention. As discussed above, U.S. Pat. Nos. 5,527,695, 5,744,336, 5,910,415, and 6,110,736 disclose a method for the site-specific transformation of plants. Using this or similar technology, a skilled practitioner, based upon the guidance provided in the instant disclosure, could transform a target plant with a construct bound by a suitable pair of recombinase excision recognition sites. This construct may or may not further comprise transgenes of interest, depending on how many steps in the step-wise transformation are utilized. Successive homologous transformation events will then build a construct within the transformed genome comprising the necessary sequence elements as disclosed and described herein. As for the functioning of the present invention, it is immaterial whether the cassettes are constructed in one piece before transformation of the plant, or are assembled in vivo through such a step-wise transformation procedure.

In an alternative embodiment, the present invention provides a gene cassette for the reversible introduction of heterologous DNA sequences into a genome of a vegetatively propagated plant, the gene cassette comprising (a) a DNA sequence for one or more genes that express a recombinase-type protein; (b) a DNA sequence for one or more transiently-active promoters operably linked to the one or more DNA sequences; (c) one or more pairs of DNA excision sequences, wherein the excision sequences are each cleavable by the recombinase-type protein; and (d) one or more heterologous DNA sequences capable of conferring a desirable phenotypic trait into the plant into which the cassette is introduced, wherein the one or more heterologous sequences are flanked by at least one of the pairs of excision sequences. Preferably, the one or more pairs of excision sequences are recognized only by the recombinase-type protein expressed by the recombinase gene of the cassette. Also, this embodiment of the present invention contemplates that the cassette may further comprise one or more selectable marker DNA sequences, such as the gene conferring resistance to the antibiotic kanamycin.

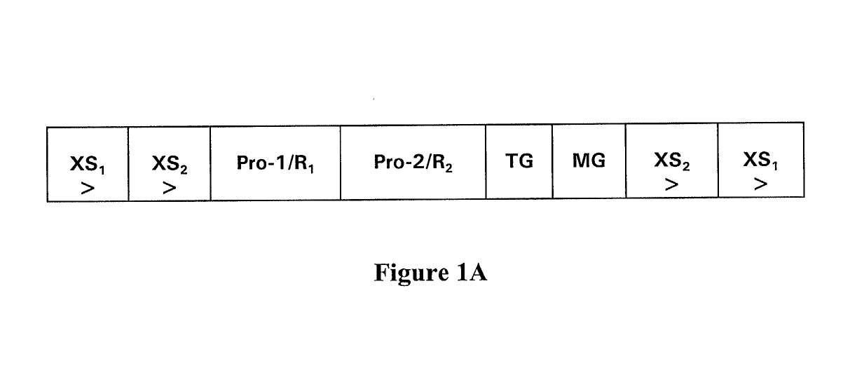

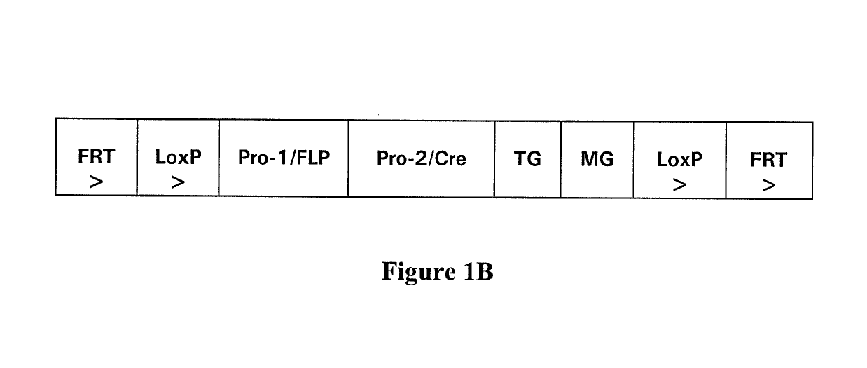

Turning to FIG. 1A, there is provided, in schematic form, the general structural elements of an exemplary cassette of the present invention. In FIG. 1A, reading from left to right, XS₁ refers to the first of a pair of excision recognition sites specific to a given recombinase protein. In a similar manner, the element XS₂ refers to the first of a pair of second, different excision recognition sites specific to a second, different, recombinase protein. Pro-1 refers to a first transiently active promoter sequence; R₁ refers to a gene sequence expressing a first recombinase protein. The combined element Pro-1/R₁ is a fusion of the sequences for the promoter and the recombinase gene. In a similar fashion, Pro-2 and R₂ refer to sequences for a second transiently-active promoter and a gene expressing a second recombinase protein, respectively. Likewise, the combined element Pro-2/R₂ is a fusion of the sequences for the second promoter and second recombinase gene. TG refers to a trait gene whose presence in the transformed plant confers a desirable phenotypic trait onto the plant. MG is a marker gene used to select those cells successfully transformed with the heterologous DNA construct of the present invention. Finally, the last two elements of the cassette, XS₁ and XS₂, are the second occurrences of the two different excision recognition site sequences that must flank the heterologous gene sequences to insure excision of the cassette upon activation and expression of the recombinase genes.

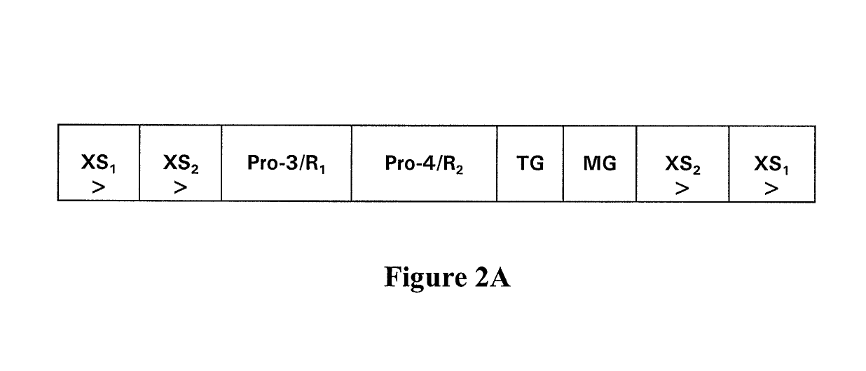

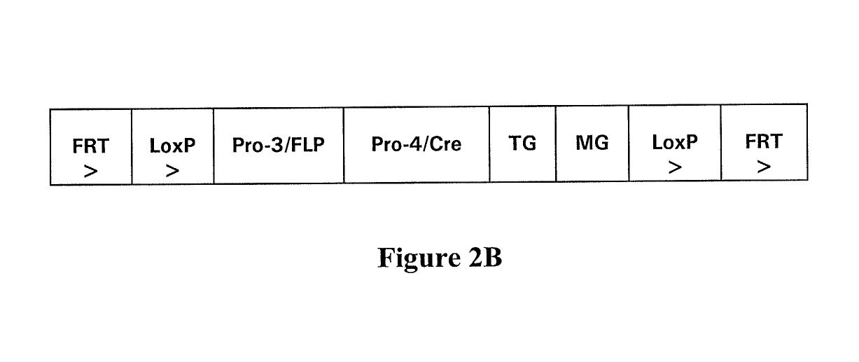

Referring to FIG. 2A, there is provided a schematic for the general elements of an alternative embodiment of the gene cassettes of the present invention. Consistent with conventions employed in FIG. 1A, Pro-3 and Pro-4 refer to third and fourth different promoter sequences whose presence in the construct is to regulate expression of the genes expressing the recombinase proteins.

In this embodiment, the disclosed invention provides that the promoter sequence is activated only in response to internal stimuli or signals such as those provided in certain organs of the plant, or associated with specific stages in the plant's developmental cycle. Alternatively, the transiently-activated promoters of the gene cassette of the present invention may be activated in response to an external or environmental stimulus. Such an external stimulus may comprise, for example, exposure to a specific chemical species. Without limitation, and as would be recognized by one of skill in the appropriate art, the chemical stimulus may comprise exposure to ethanol, ecdysone, a glucocorticoid such as dexamethasone (DEX), antobiotics such as tetracycline, forms of estrogen such as estradiol, or heavy metals such as cadmium (Cd²⁺) and copper (Cu²⁺). In addition, the transiently-activated promoter of the gene cassette of the present invention may be activated in response to, or in combination with, other external stimuli such as heat shock, exposure to electromagnetic radiation, or exposure to reduced temperatures. The present invention also provides a gene cassette where activation of the selectively-activated promoter is achieved only in response to external stimuli, and even then only in certain organs or tissues of the plant, some of which may exist only at specific stages in the plant's developmental cycle.

One of skill in the appropriate art would readily recognize that a number of specific examples of externally inducible promoter systems are available for use on the practice of the present invention. Provided with the guidance of the instant disclosure and readily available information such as that found in the appropriate technical literature, any number of externally inducible systems could be utilized in the present invention. By way of example, and without limitation to the scope of the claimed invention, these promoter systems would include the following:

glucocorticoid inducible promoter: comprises a promoter derived from pathogenic strains of Pseudomonas syringae pv. tomato carrying the avrRpt2 avirulence gene (McNellis, T. W., et al., “Glucocorticoid-inducible expression of a bacterial avirulence gene in transgenic Arabidopsis induces hypersensitive cell death,” Plant Journal 14: 247-257 (1998); Kang, H. G., et al., “A glucocorticoid-inducible transcription system causes severe growth defects in Arabidopsis and induces defense-related genes,” Plant Journal 20: 127-133 (1999); Aoyama, T. and Chua, N. H., “A glucocorticoid-mediated transcriptional induction system in transgenic plants,” Plant J. 11:605-12 (1997));

tetracycline-inducible promoter: roIC activity (from Agrobacterium rhizogenes) on endogenous cytokinin conjugates demonstrated that transcription and expression of the gene was under the control of a tetracycline-inducible promoter (Faiss, M., et al., “Chemically induced expression of the roIC-encoded beta-glucosidase in transgenic tobacco plants and analysis of cytokinin metabolism: roIC does not hydrolyze endogenous cytokinin glucosides in planta,” Plant J. 10: 33-46 (1996); Gatz, C. et al., “Stringent repression and homogeneous de-repression by tetracycline of a modified CaMV 35S promoter in intact transgenic tobacco plants,” Plant J. 2: 397-404 (1992); Faryar, K. and Gatz, C., “Construction of a Tetracycline-inducible promoter in Schizosaccharomyces pombe,” Curr. Genet. 21: 345-349 (1992));

estrogen-inducible promoter: gene expression in cells of Black Mexican Sweet (BMS) maize inbred demonstrated under control of promoter comprising multiple repeats of an estrogen receptor binding site (Bruce, W., et al., “Expression profiling of the maize flavonoid pathway genes controlled by estradiol-inducible transcription factors CRC and P,” Plant—Cell. 12: 65-79 (2000));

heavy metal-inducible promoters: 35S CaMV-derived promoter demonstrated to control expression of beta-glucoronidase in tobacco in presence of Cd²⁺ (Brandle, J. E., et al, “Field performance and heavy metal concentrations of transgenic flue-cured tobacco expressing a mammalian metallothionein-beta-glucuronidase gene fusion,” Genome 36: 255-260 (1993)); regulation mechanism from yeast metallothionein (MT) gene derived from 35S CaMV promoter controls expression of beta-glucoronidase (GUS) reporter gene in transgenic plants in response to exposure to Cu²⁺ (Mett, V. L., et al., “Copper-controllable gene expression system for whole plants,” Proc Natl Acad Sci U S A 90: 4567-71 (1 993));

gosmotic stress-inducible: gene expression in soybean plant demonstrated to be under control of inducible heat shock promoter (IHSP) responsive to mannitol stress (De Ronde, J. A., et al., “Effect of antisense L-delta1-pyrroline-5-carboxylate reductase transgenic soybean plants subjected to osmotic and drought stress,” Plant Growth Regul.32: 13-26 (2000));

low temperature-inducible promoter: cor15a gene of Arabidopsis thaliana demonstrated to be under the control of cold inducible promoter located in 5′ region between −305 and +78 (Dordrecht, “The 5′-region of Arabidopsis thaliana cor15a has cis-acting elements that confer cold-, drought- and ABA-regulated gene expression,” Plant Mol. Biol. 24: 701-713 (1994)).

A number of other external stress-induced promoters have been reported in the literature. Consistent with the practice of the present invention, one of skill in the appropriate art, based upon knowledge imputed thereto and the specific disclosures of the present application, would be able to construct appropriate gene cassettes comprising a variety of externally-inducible regulatory elements to achieve the desired ends of the present invention.

Preferably, the gene sequence of the cassette expresses a protein of the type selected from the group consisting of recombinases, invertases, integrases, transposases and resolvases. More preferably, the sequence expresses a recombinase-type protein selected from the group consisting of FLP, Cre, R, Gin, PIV, C31, FimB, KW, SSV, IS1110/IS492, ParA, TnpX, and others as discussed above.

Furthermore, the gene cassette of the present invention comprises one or more promoter gene sequences selected from the group consisting of organ- or developmental stage-specific gene promoters selected from the group consisting of seed-, fruit-, pollen-, stem/shoot-, leaf-, root-specific gene promoters. Preferably, the one or more promoter gene sequences are selected from the group consisting of AG, AGL5, Bcp1, LAT52, PLENA, SIM, avrRpt2, alc and others discussed above.

The expression of a gene is primarily directed by its own promoter, although other DNA regulatory elements are necessary for efficient expression of a gene product. Promoter sequence elements include the TATA box consensus sequence (TATAAT), which is usually 20 to 30 base pairs (bp) upstream of the transcription start site. In most instances the TATA box is required for accurate transcription initiation. By convention, the transcription start site is designated +1. Sequences extending in the 5′ (upstream) direction are given negative numbers and sequences extending in the 3′ (downstream) direction are given positive numbers.

Promoters can be either constitutive or inducible. A constitutive promoter controls transcriptions of a gene at a constant rate during the life of a cell, whereas an inducible promoter's activity fluctuates as determined by the presence (or absence) of a specific inducer. The regulatory elements of an inducible promoter are usually located further upstream of the transcriptional start site that the TATA box. Ideally, for experimental purposes, an inducible promoter should possess each of the following properties: a low to nonexistent basal level of expression in the absence of inducer, a high level of expression in the presence of inducer, and an induction scheme that does not otherwise alter the physiology of the cell.

Among the promoter sequences available for use in the cassettes of the present invention, the following are provided by way of example only, and not to limit in any way the scope of the claimed invention. These include the AG (AGAMOUS) gene promoter, which is active specifically in the floral meristem that gives rise to stamens and carpels, and continues to be active in stamens and carpels until after fertilization. The AGL5 (“AGL5”—AGAMOUS-like, No. 5) promoter becomes active in all cell types of carpel primordia including ovule primordia and mature ovules. The promoters of PLENA of snapdragon and SLIM of white campion are specifically active in the floral meristem that gives rise to stamens and carpels, and in developing carpels and stamens, similar to the AG gene promoter. The Bcp1 gene promoter cloned from Brassica is specifically active in anther with high activities in both the haploid pollen and diploid tapetum. The LAT52 gene promoter is isolated from tomato plants. LAT52 is specifically active in pollen and anthers with no activity in other floral organs or non-reproductive tissues.

In addition to promoters that are active only in specific organs or tissues of plants, there are other promoters that are active in response to external stimuli, as indicated above. For example, CT/CI is an artificial chemically-inducible system that contains two transcription units. The first unit employs a constitutive promoter (e.g., 35S CaMV, from the cauliflower mosaic virus) to express a chemical-responsive transcription factor, whereas the second unit comprises multiple copies of transcription factor binding sites linked to a minimal plant promoter (e.g., a truncated 35S CAMV promoter). The second unit is used to express the target gene. In the case of the ecdysone-inducible system, the transcription factor contains the DNA binding domain of a glucocorticoid receptor and the ecdysone regulatory domain of the Heliothis virescens ecdysone receptor. It has been shown that, in transgenic plants, an ecdysone agonist can induce the expression of a target gene over 400-fold. The system is highly responsive to RH5992, a non-steroidal ecdysone agonist that lacks phytotoxicity and is currently used as lepidopteran control agent on a wide range of crops. In the case of ethanol-inducible system, the intact Aspergillus nidulans AlcR activator has been used to control expression of the target gene in plants. With the ethanol inducible system, high levels of target gene expression in transgenic plants have been observed upon ethanol induction. Pending development of non-volatile inducers, the ethanol-inducible system appears to be an ideal system for field applications.

In another embodiment, the present invention provides a gene cassette for the reversible introduction of heterologous DNA sequences into a genome of a sexually propagated plant, the gene cassette comprising (a) a first DNA sequence comprising (i.) a sequence that expresses a first recombinase-type protein; (ii.) a sequence for an inducible promoter, operably linked to the sequence that expresses the first recombinase-type protein, wherein the promoter is capable of being activated in reaction to an external stimulus; (iii.) a sequence that expresses a transcription factor capable of regulating transcriptional activity of the sequence that expresses the first recombinase-type protein under control of the sequence for the externally inducible promoter; and (iv.) one or more pairs of DNA excision site sequences wherein the excision sites are capable of being cleaved only by the first recombinase-type protein; (b) a second DNA sequence comprising (i.) a sequence capable of expressing a second recombinase-type protein; (ii.) a sequence for a transiently-active promoter capable of controlling expression of the second recombinase-type protein; and (iii.) one or more heterologous DNA sequences capable of conferring a desirable phenotypic trait on the genome into which the cassette is introduced; and (c) one or more pairs of DNA excision site sequences wherein the excision sites are capable of being cleaved only by the second recombinase-type protein.

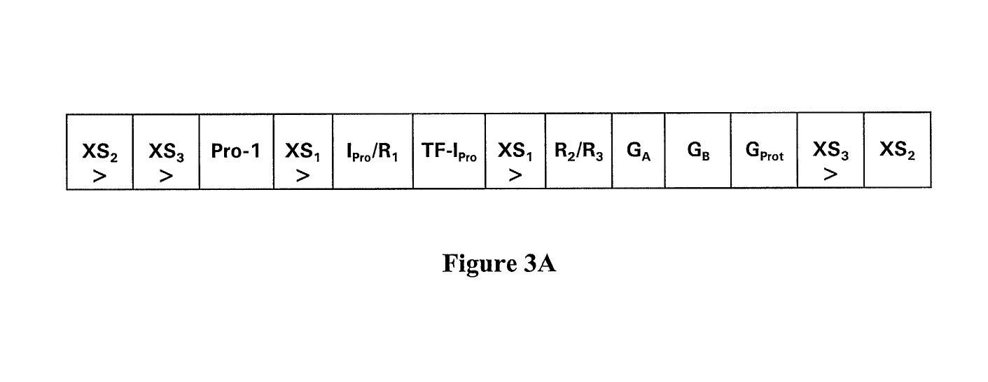

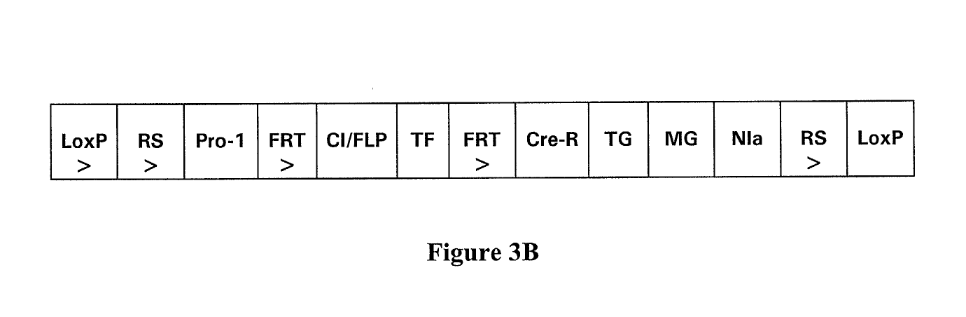

Referring now to FIG. 3A, there is provided, in schematic form, the general structural elements of an exemplary cassette of this embodiment of the present invention. In FIG. 3A, consistent with the conventions employed with FIGS. 1A and 2A, XS₁ refers to the first of a pair of excision recognition sites specific to a given recombinase protein. In a similar manner, the element XS₂ refers to the first of a pair of second, different excision recognition sites specific to a second, different, recombinase protein. The element XS₃ refers to the first of a pair of third, different excision recognition sites specific to a third, different, recombinase protein. Pro-1 refers to a first transiently active promoter sequence. I_{Pro} refers to a transiently-active promoter sequence whose activity is inducible in response to external stimuli; R₁ refers to a gene sequence expressing a first recombinase protein. The combined element I_{Pro}/R₁ is a fusion of the sequences for the inducible promoter and the first recombinase gene. TF refers to a transcription factor, itself normally comprising a constitutively active promoter such as the 35S CaMV promoter, that serves to regulate functioning of the inducible promoter. Only in the presence of both the transcription factor and the inducible promoter will the first recombinase gene express the first recombinase protein. R₂ and R_{S} refer to second and third sequences for different recombinase genes. The combined element R₂/R₃ is a fusion of the DNA sequences for the second and third recombinase genes. G_{A} and G_{B} refer to first and second gene sequences where the genes may confer a desirable phenotypic trait onto the plant into which the cassette of the present invention is transformed, or may be selectable marker genes. G_{Prot} refers to a gene sequence for a protease protein that is capable of recognizing a specific amino acid sequence. Upon activation and expression of the protease gene, the resulting protease protein will cleave a specific site within an amino acid sequence. In this embodiment of the present invention, the combined element R₂/R_{S} encodes a polypeptide with individual elements linked by an amino acid sequence recognizable by the protease protein encoded by the protease gene of the cassette. Upon cleavage of the recognition sequence linking the R₂ and R₃ polypeptide elements, active forms of the separate recombinase proteins result. These, in turn, function to excise the sequence between their respective excision recognition sites. Finally, XS₃ and XS₁ refer to the second members of the pairs of different recombinase recognition sequence sites.

This embodiment of the present invention further contemplates that the first DNA sequence is capable of blocking the induction of expression of the second recombinase-type protein by the transiently-active promoter. The gene cassette of the present embodiment further contemplates that the second DNA sequence comprises (a) a sequence capable of expressing a third recombinase-type protein; (b) a sequence capable of expressing a protease protein; and (c) one or more pairs of DNA excision site sequences wherein the excision sites are capable of being cleaved only by the third recombinase-type protein, wherein the third recombinase-type protein is linked to the second recombinase-type protein through a specific amino acid sequence capable of being cleaved by the protease protein. Preferably, the protease protein is NIa. The gene cassette also provides that the second DNA sequence further comprises a second promoter sequence operatively linked to the sequence that expresses the protease protein. Furthermore, the amino acid sequence linking the second recombinase-type protein to the third recombinase-type protein comprises the following amino acid sequence: V-R-T-Q-G-P-K-R.

Nla is a site-specific recognition and cleavage protease. NIa recognizes V-R-T-Q/G-P-K-R sequence and cleaves at the Q/G position. NIa has been successfully used to produce three gene products using a single gene promoter in higher plants.

This embodiment provides that the second DNA sequence further comprises a marker gene sequence that, preferably, confers resistance to an antibiotic such as kanamycin. It is also preferred that the transiently-active promoter sequence is activated only in certain organs of the plant or, alternatively, only at specific stages in the plant's developmental cycle. As a further alternative, the externally-inducible promoter sequence is activated in response to an external or environmental stimulus. Such an external stimulus may comprise, for example, exposure to a specific chemical species. Without limitation, and as would be recognized by one of skill in the appropriate art, the chemical stimulus may comprise exposure to ethanol, ecdysone, a glucocorticoid such as dexamethasone (DEX), antobiotics such as tetracycline, forms of estrogen such as estradiol, or heavy metals such as cadmium (Cd²⁺) and copper (Cu²⁺)

In addition, the transiently-activated promoter of the gene cassette of the present invention may be activated in response to, or in combination with, other external stimuli such as heat shock, exposure to electromagnetic radiation, or exposure to reduced temperatures. The present invention also provides a gene cassette where activation of the selectively-activated promoter is achieved only in response to external stimuli, and even then only in certain organs or tissues of the plant, some of which may exist only at specific stages in the plant's developmental cycle.

Preferably, the gene sequence of the cassette of this embodiment of the present invention expresses a protein of the type selected from the group consisting of recombinases, invertases, integrases, transposases and resolvases. More preferably, the sequence expresses a recombinase-type protein selected from the group consisting of FLP, Cre, Gin, R, PIV, C31, FimB, KW, SSV, IS1110/IS492, ParA, and TnpX.

Furthermore, the gene cassette of the present invention comprises one or more promoter gene sequences selected from the group consisting of organ- or developmental stage-specific gene promoters selected from the group consisting of seed-, fruit-, pollen-, stem/shoot-, leaf-, root-specific gene promoters. Preferably, the one or more promoter gene sequences are selected from the group consisting of AG, AGL5, Bcp1, LAT52, PLENA, SIM, avrRpt2, and alc.

### EXAMPLES

### Example 1

### A Gene Cassette for the Reversible Introduction of Heterologous DNA Sequences into a Genome of a Vegetatively Propagated Plant

Referring now to FIG. 1B, the following describe the individual elements of the schematic of the gene cassette of this Example:

FRT is the recognition sequence of FLP;

LoxP is the recognition sequence of Cre;

Pro-1 and Pro-2 are two distinct pollen specific promoter sequences, such as Bcp1 and LAT52;

FLP is a recombinase that recognizes FRT sequences and excises the DNA sequence flanked by two FRT sequences that orient as direct repeats;

Cre is a recombinase that recognizes LoxP sequences and excises the DNA sequence flanked by two loxP sequences that orient as direct repeats;

TG is a trait gene(s), such as herbicide-resistance gene, insect- or disease- resistance genes;

MG is a marker gene, such as kanamycin resistance gene, for selection of transgenic cells and plants.

A site-specific DNA recombinase excises DNA sequences flanked with two specific sequences (direct repeats) recognizable by the recombinase. When transgenic plants reach a specific developmental stage, a recombinase gene that is under the control of an organ- and developmental stage-specific gene promoter is expressed. As a result, all transgenes flanked by the two excision sequences (recognized by the recombinase) are excised. A safeguard system (a second excision system), comprising a different DNA recombination system, is implemented to ensure complete excision of transgenes from the host genome. The plant-active gene promoters that drive expression of the two recombinase genes control when (at what developmental stage) and where (in what organs) the transgenes are excised from the host genome.

When transgenic plants reach their reproductive stage, the FLP gene that is under the control of Pro-1 (a pollen-specific gene promoter such as Bcp1 of Brassica) is expressed specifically in the pollen. As a result, all transgenes flanked by the two FRT sequences are excised. Although the efficiency of the FLP-mediated excision is greater than 98%, a safeguard system (a second excision system), preferably the loxP/Cre system, is implemented. Expression of the Cre gene is controlled by Pro-2, another pollen specific gene promoter. Pro-2 can be the LAT52 gene promoter from tomato. The Cre protein excises the transgenes flanked by the two loxP sequences. With these independent excision systems, complete or near complete removal of the transgenes from pollen is achieved.

This gene cassette is designed to remove all transgenes from the pollen of vegetatively propagated transgenic plants. With this gene cassette, the pollen produced from transgenic plants becomes essentially wild-type (transgene free). Cross-pollination of transgenic plants with wild-type plants can cause spread of transgenes to the environment. With this gene cassette, undesirable spread of transgenes via cross-pollination can be reduced or eliminated.

### Example 2

### A Second Gene Cassette for the Reversible Introduction of Heterologous DNA Sequences into a Genome of a Vegetatively Propagated Plant

Referring now to FIG. 2B, the following describe the individual elements of the schematic of the gene cassette of this Example:

FRT is the recognition sequence of FLP;

LoxP is the recognition sequence of Cre;

Pro-3 is a promoter sequence that is specifically active in male (stamens including pollen) and female organs (carpels) of the plant; Pro-3 is inactive after fertilization; an example of pro-3 is AG gene promoter of Arabidopsis;

FLP is a recombinase that recognizes FRT sequences and excises the DNA sequence flanked by two FRT sequences that orient as direct repeats;

Cre is a recombinase that recognizes LoxP sequences and excises the DNA sequence flanked by two loxP sequences that orient as direct repeats;

Pro-4 is a promoter sequence that is specifically active in carpels and stamens; examples of Pro-4 are AGL5 of Arabidopsis, PLENA of snapdragon, and SIM of white campion;

TG is a trait gene(s), such as herbicide-, insect- or disease-resistance genes;

MG is a marker gene, such as the kanamycin resistance gene, useful for selection of transgenic cells and plants.

A site-specific DNA recombinase excises DNA sequences flanked with two specific sequences (two direct repeats) recognizable by the recombinase. When transgenic plants reach a specific developmental stage, a recombinase gene that is under the control of an organ- and developmental stage-specific gene promoter is expressed. As a result, all transgenes flanked with the two excision sequences (recognized by the recombinase) are excised. A safeguard system (a second excision system), a second, different DNA recombination system, is implemented to ensure complete excision of transgenes from the host genome. The plant-active gene promoters that drive expression of the two recombinase genes control when (at what developmental stage) and where (in what organs) the transgenes are excised.

When transgenic plants reach their reproductive stage, the FLP gene that is under the control of Pro-3 (e.g., the AG promoter) is expressed specifically in stamens and carpels. As a result, all transgenes flanked with the two FRT sequences are excised. Although the efficiencies of the FLP-mediated excision are greater than 98%, a safeguard system, that is, a second, different site-specific excision system, such as the loxP/Cre system, is implemented. Expression of the Cre gene is driven by Pro-4, a carpel- and stamen-specific gene promoter, such as AGL5 of Arabidopsis, PLENA of snapdragon or SIM of white campion. The Cre protein excises the transgenes flanked by the two loxP sequences. With these two-excision systems working independently, complete or near-complete removal of the transgenes from pollen, fruits and seeds is achieved.

This gene cassette is designed to remove all transgenes from the pollen, fruits and seeds of vegetatively propagated transgenic plants. With this system, non-transgenic pollen, seeds and fruits are produced from transgenic plants. This gene cassette reduces or eliminates potential health implications of GM plants when seeds and fruits produced from transgenic plants are used as food for human and animals. At the same time, this system eliminates undesirable spread of transgenes to the environment via pollen (cross pollination with wild-type plants) and seeds.

### Example 3

### A Gene Cassette for the Reversible Introduction of Heterologous DNA Sequences into a Genome of a Sexually Propagated Plant

Referring now to FIG. 3B, the following describe the individual elements of the schematic of the gene cassette of this Example:

LoxP is the recognition sequence of Cre;

RS is the recognition sequence of R;

Pro-1 is a promoter sequence that is active specifically in the pollen and carpels but inactive after fertilization. An example of Pro-1 is the AG (AGMOUS) gene promoter of Arabidopsis;

FRT is the recognition sequence of FLP;

CI is an artificial chemical-inducible gene promoter that can be activated by external applied chemical. Examples of the CI are an ethanol-inducible gene promoter and an ecdysone-inducible gene promoter;

FLP is a recombinase that recognizes FRT sequences and excises the DNA sequence flanked by two FRT sequences that orient as direct repeats;

TF is a transcription factor specific for CI. The presence of both TF and its inducer activates CI;

Cre-R is a fusion of the coding sequences of the Cre and R genes; Cre is a recombinase that recognizes LoxP sequences and excises the DNA sequence flanked by two loxP sequences that orient as direct repeats; R is a recombinase that recognizes RS sequences and excises the DNA sequence flanked by two RS's that orient as direct repeats; the Cre and R coding sequences are linked with a specific recognition sequence for a specific protease, such as V-R-T-Q/G-P-K-R for NIa;

TG is a trait gene(s). Examples are herbicide-resistance gene, insect-or disease- resistance genes;

MG is a marker gene, such as kanamycin-resistance gene, useful for selection of transgenic cells and plant;

NIa is a site-specific cleavage protease.

The first gene in this gene cassette is a fusion of a chemical-inducible gene promoter and a coding sequence of a DNA recombinase gene. The second gene is a chimeric gene containing an organ- and developmental stage-specific gene promoter (Pro-1), a blocking sequence (the first gene is used as a blocking sequence), and a fusion of the coding sequences of the second and third recombinase genes. Because the blocking sequence contains two specific excision sequences recognizable by the first recombinase, the blocking sequence is excised when the first recombinase gene is expressed upon application of the chemical. Consequently, Pro-1 and the fusion of the coding sequences of the second and third recombinase genes are operably linked. The second and third recombinases themselves are connected as translational fusions with an excision sequence recognizable by a site-specific protease. All transgenes including a marker gene, trait genes and DNA recombinase genes are flanked with the specific excision sequences recognizable by the two recombinases. Upon expression of the second and third DNA recombinase genes at a specific developmental stage, all transgenes are excised from the genome of specific organs.

In practice, if dry seeds (before planting) are treated with a chemical inducer that specifically activates the chemical-inducible gene promoter, the first recombinase gene is expressed during seed germination. If young seedlings in the field are treated with the chemical inducer (it can be sprayed together with a herbicide in the field), expression of the first recombinase gene is induced in the field. In both cases, the DNA sequences flanked by the two excision sequences (recognized by the first recombinase) are excised from the genome. As a result of the excision, Pro-1 is operably linked with the DNA sequence of the second and third site-specific recombinase genes. When Pro-1 becomes active at a specific developmental stage, the fusion protein of the second and third recombinases is produced in specific organs. The protease that is expressed constitutively in the cell cleaves the fusion protein into two pieces to produce two different functional recombinases. The plant-active gene promoter that drives expression of the second and third recombinase genes controls when (developmental stage) and where (organs) the transgenes are excised from the genome. Because two different types of recombinases are used, complete or near-complete removal of transgenes is achieved.

If dry seeds (before planting) are treated with a chemical inducer that specifically activates CI, expression of FLP is then induced during seed germination. If, alternatively, young seedlings in the field are treated with the chemical inducer (it can be sprayed together with a herbicide in the field), expression of FLP is induced. Externally applied chemical inducers can be ethanol if CI is an ethanol-inducible gene promoter, or an ecdysone if CI is an ecdysone-inducible gene promoter. In both cases, upon application of the chemical, the DNA sequences flanked by the two FRT sequences are excised. After the excision, AG and Cre-R are operably linked. When AG becomes active at the reproductive stage, the Cre-R fusion protein is produced specifically in stamens and carpels. Because Cre and R are connected as translational fusions with V-R-T-Q/G-P-K-R (a recognition sequence of the protease, Nla), NIa cleaves the fusion protein into two pieces to produce functional Cre and R recombinases. A constitutive and strong gene promoter such as 35S CaMV gene promoter is used to drive expression of the NIa gene. Because both Cre and R proteins are present in the cell, transgenes in stamens and carpels (i.e., pollen, seeds and fruits) are efficiently excised.

Gene Cassette C is designed to remove all transgenes from the pollen, fruits and seeds of sexually propagated transgenic plants. With this gene cassette, non-transgenic products (e.g., fruits and seeds) can be produced from transgenic plants. The system reduces/eliminates potential health implications of transgenes if transgenic plants or their organs (e.g., seeds and fruits) are used as food for humans and animals. At the same time, this gene cassette eliminates undesirable spread of transgenes to the environment via pollen (cross pollination with wild-type plants) and seeds. Furthermore, the system can also be used to remove transgenes from other organs if AG is replaced with an appropriate gene promoter.

What is claimed is:

1. A method for the creation of a transiently transgenic plant whereby a heterologous transgene temporarily conveys a desirable phenotypic trait to the plant, the method comprising the following steps:
a) constructing a gene cassette comprising:
(i.) one or more DNA sequences for a gene conferring a desirable phenotypic trait;
(ii.) one or more DNA sequences expressing a recombinase-type protein;
(iii.) at least one pair of DNA excision sequences cleavable by the recombinase-type protein, wherein the excision sequences flank the heterologous DNA; and
(iv.) a transiently activated promoter operably linked to the DNA sequence expressing the recombinase-type protein and controlling expression of the protein, wherein the promoter is activated, and thereby directs expression of the recombinase-type protein, in response to developmental or external stimuli;
b) introducing the cassette into the genome of the plant; and
c) exposing the DNA sequences within the cassette to a stimulus that activates the promoter, whereby the promoter directs expression of the recombinase protein, and the recombinase protein excises the heterologous DNA from the genome of the plant.

2. The method of claim 1, wherein the gene cassette further comprises a DNA sequence for a marker gene.

3. The method of claim 1, wherein the transiently-active promoter is activated only in certain organs of the plant.

4. The method of claim 1, wherein the promoter sequence is activated only at specific stages in the plant's developmental cycle.

5. The method of claim 1, wherein the promoter sequence is activated in response to an external stimulus.

6. The method of claim 5, wherein the external stimulus is selected from the group consisting of exposure to a specific chemical species, heat shock, exposure to electromagnetic radiation, and exposure to reduced temperatures.

7. A gene cassette for the reversible introduction of heterologous DNA sequences into a genome of a vegetatively propagated plant, the gene cassette comprising:
a) a DNA sequence for one or more genes that express a recombinase-type protein;
b) a DNA sequence for one or more transiently-active promoters operably linked to the one or more DNA sequences;
c) one or more pairs of DNA excision sequences, wherein the excision sequences are each cleavable by the recombinase protein; and
d) one or more heterologous DNA sequences capable of conferring a desirable phenotypic trait on the plant into which the cassette is introduced, wherein the one or more heterologous sequences are flanked by at least one of the pairs of excision sequences.

8. The gene cassette of claim 7, wherein the one or more pairs of excision sequences are recognized only by the recombinase-type protein expressed by the recombinase gene of the cassette.

9. The gene cassette of claim 7, wherein the cassette further comprises one or more selectable marker DNA sequences.

10. The gene cassette of claim 7, wherein the promoter sequence is activated only in certain organs of the plant or only at specific stages in the plant's developmental cycle.

11. The gene cassette of claim 7, wherein the promoter sequence is activated by exposure of the transformed plant to an external stimulus.

12. The gene cassette of claim 11, wherein the external stimulus is selected from the group consisting of exposure to a specific chemical species, osmotic stress, heat shock, exposure to electromagnetic radiation, and exposure to reduced temperatures.

13. The gene cassette of claim 7, wherein the gene sequence expresses a recombinase-type protein selected from the group consisting of recombinases, invertases, integrases, transposases and resolvases.

14. A gene cassette for the reversible introduction of heterologous DNA sequences into a genome of a sexually propagated plant, the gene cassette comprising:
a) a first DNA sequence comprising:
(i.) a sequence that expresses a first recombinase-type protein;
(ii.) a sequence for an inducible promoter, operably linked to the sequence that expresses the first recombinase-type protein, wherein the promoter is capable of being activated in reaction to an external stimulus;
(iii.) a sequence that expresses a transcription factor capable of regulating transcriptional activity of the sequence that expresses the first recombinase-type protein; and
(iv.) one or more pairs of DNA excision site sequences wherein the excision sites are capable of being cleaved only by the first recombinase-type protein;
b) a second DNA sequence comprising:
(i.) a sequence capable of expressing a second recombinase-type protein;
(ii.) a sequence for a transiently-active promoter capable of controlling expression of the second recombinase protein; and
(iii.) one or more heterologous DNA sequences capable of conferring a desirable phenotypic trait on the plant into the genome of which the cassette is introduced; and
c) one or more pairs of DNA excision site sequences wherein the excision sites are capable of being cleaved only by the second recombinase-type protein.

15. The gene cassette of claim 14, wherein the first DNA sequence is capable of blocking the induction of expression of the second recombinase-type protein by the transiently-active promoter.

16. The gene cassette of claim 14, wherein the second DNA sequence further comprises:
a) a sequence capable of expressing a third recombinase-type protein;
b) a sequence capable of expressing a protease protein; and
c) one or more pairs of DNA excision site sequences wherein the excision sites are capable of being cleaved only by the third recombinase-type protein,
wherein the third recombinase-type protein is linked to the expressing the second recombinase-type protein through a specific amino acid sequence capable of being cleaved by the protease protein.

17. The gene cassette of claim 14, wherein the second DNA sequence further comprises a second promoter sequence operatively linked to the sequence that expresses the protease protein.

18. The gene cassette of claim 14, wherein the second DNA sequence further comprises a marker gene sequence.

19. The gene cassette of claim 14, wherein the transiently-active promoter sequence is activated only in certain organs of the plant or only at specific stages in the plant's developmental cycle.

20. The gene cassette of claim 14, wherein the externally- inducible promoter sequence is activated in response to exposure to an external stimulus.

21. The gene cassette of claim 20, wherein the external stimulus is selected from the group consisting of exposure to a specific chemical species, heat shock, exposure to electromagnetic radiation, and exposure to reduced temperatures.

22. The gene cassette of claim 14, wherein the gene sequences expressing a recombinase-type protein express a protein selected from the group consisting of recombinases, invertases, integrases, transposases and resolvases.


SEQUENCE LISTING (11 sequences)

SEQ ID NO: 1 - DNA, 33 bp, Bacteriophage P1
ataacttcgtatatgtatgctatacgaagttat

SEQ ID NO: 2 - DNA, 1553 bp, Bacteriophage P1
tgcgcagctggacgtaaactcctcttcagacctaataacttcgtatagcatacattatacgaagttatattaagggttattgaatatgatcaatttacctgtaaatccatacagttcaataccttagcaggtcaaatagtgaccacttgatcatttgatcaaggttgcgctacgtaaaatctgtgaaaaattggcggtgttagtcctacagatttcgcgtaccacttagcaccaccaatcaatcagaggtgaaaaatgggatattcaactgctaaagtgtccactcatcttgagcttgagaaaaaccgtggttactggcgggcaaaagggtttgatcgtgatagttgccaactgtcattatcgcgcggtgaagagaaaatagaacgcacgcgcggtcgctggcgtttctatgacgagaaccataaacaggtaaaggcagagccgatcctgtacactttacttaaaaccattatctgagtgttaaatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgctataattagttgata

SEQ ID NO: 3 - DNA, 61 bp, Saccharomyces cerevisiae
gggctaccatggagaagttcctattccgaagttcctattctctagaaagtataggaacttc

SEQ ID NO: 4 - DNA, 1272 bp, Saccharomyces cerevisiae
atgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataa

SEQ ID NO: 5 - DNA, 900 bp, Brassica napus
tatcattcctttaatttcaaggaattatagaacaaaaaatgttcttataaaaattaagaaaggaacaagggattcattcctactattctgtgcttggtcattattttcctcttcattcatattgtttctttaattgttaccaattagaactttaacgaataaatagttaattcgtattatgagatttacacaattcttattcactcaatttggagttttaaagattttttaaaagatttatggtgggaaccttcttcttttcttatttatcatgatgatgataaccttcccagcagaattattcttagaactttttttcacatttaggtatccatgcctaagtaaggcttagttaaagatgttttataaactttgatcaaaatattcattcaattaatttgagcttcaactataaattgttgtatgcattcttttagcctgtaagatatcagacattcacgtttcgatattcatcaaacaagtatataaataatatgaatattgtacattcattttattcggaccaaaaaaaataaaaataaatattcgtattcatctatgctttggcatggtccgttcttttttcttgattggctcgttaccattcaaaaatatataccttagcaaacccatttttagacattccagttgatctacattagattgaacggtattcctcctacgtagtaagaacgttttctatttttctttgtttcagtcatacaacacaactatatatacacagcaaccccatctcctctccaatcatcacaatctctaacgttaaaccctaagacaaactaaaagagagctacgtacaaggagacagagagaagaatgggtcgccaaaacgctgtcgtagtttttggccttgtgttcttggccatccttggcctcgcc

SEQ ID NO: 6 - DNA, 1048 bp, Arabidopsis thaliana
gaaaatgatgaggaatgggcaaaacacaaaagagtttcctttcgtaactacaattaattaatgcaaatctgagaaagggttcatggataatgactacacacatgattagtcattccccgtgggctctctgctttcatttactttattagtttcatcttctctaattatattgtcgcatatatgatgcagttcttttgtctaaattacgtaatatgatgtaattaattatcaaaataatattaacgacatgcaatgtatataggagtagggcaataaaaagaaaaggagaataaaaagggattaccaaaaaaggaaagtttccaaaaggtgattctgatgagaaacagagcccatacctctcttttttcctctaaacatgaaagaaaaattggatggtcctccttcaatgctctctccccacccaatccaaacccaactgtcttctttctttcttttttcttctttctatttgatattttctaccacttaattccaatcaatttcaaatttcaatctaaatgtatgcatatagaatttaattaaaagaattaggtgtgtgatatttgagaaaatgttagaagtaatggtccatgttctttctttctttttccttctataacacttcagtttgaaaaaaaactaccaaaccttctgttttctgcaaatgggtttttaaatacttccaaagaaatattcctctaaaagaaattataaaccaaaacagaaaccaaaaacaaaaaataaagttgaagcagcagttaagtggtactgagataataagaatagtatctttaggccaatgaacaaattaactctctcataattcatcttcccatcctcacttctctttctttctgatataattaatcttgctaagccaggtatggttattgatgatttacactttttttaaaagtttcttccttttctccaatcaaattcttcagttaatccttataaaccatttctttaatccaaggtgtttgagtgcaaaaggatttgatctatttctcttgtgtttatacttcagctagggcttatagaaatg

SEQ ID NO: 7 - DNA, 5497 bp, Arabidopsis thaliana
tatcattttttttatatgtcaatatcacaacaagtaaacatcatcaacttcaaatctcactaacataaatagtatgctaattttcttcactatctgagtaattttctccttctttttacatcattcgattctatatttgccacaccattaaatagataagtatattggttcttactactaataatcagtttagccatatatctaattacacagttttcttttatatgttactttatatttgtacaccatcagttactatatacggaataaattacacctcatcatgtaattacaatattatcataaattttttgaaattctattctttaatattttcagtatccagaatccattttcttatatttattctggcaaaataatgtaaaatggtatcaaatatacaacaccatatatttgtttataaataacttccttaattagtgaaacaaattttcctgcagaatgtcacttaaatctttaaaacaattcacaaaagaaaagggaatagagctgaaattttttcctctttaaaaagatcaagatttagtaaaaactagaaacaaagaagaagatcgatatttgttgtaacaaaaaaatgtactgatcagatttacccaaagattttagtgcctcaaaaactttcacatgaaaagcaaagacaagtaatggtaagtagagtctgcatctcatgagtgattgcccaacttgaaccctcttttcatttagacctttgtgaaagaccacacaagaactacccaccaataactctctctttttttcttcatttccaaaaactatttattcatttctacaatttctcaatttgggggtgtccttaataagtattatatttatgaaaagtccggatgtatggtgggtgttatttatggtaattaacactatttattaataatatttttcatgtgatttcaaaagctgatgtactcatgctttgtgaacctttagattgctctatgtcaatcttttattttaacctatcatatgtctaaatgtactgaaaagaaacaccagtttaattaattatacttccctcatatataactatcaaccaagtacaaaacttttgtcaattctcaaaatcaactttcaccacataattatctaacatgtgtatgttccaaaaccagtttaaatagaattacttttcagaaaatacatgtatattaactctatctaataaagaagaaacacatacttatctcatagattccattcataaaactatgctttagtgagtaagaaaaccagtaatcaaacacaaattgacaagacactatatggatgtaaaaagtggggaaaaatggtgataaatagtagagaaaattaaaaagaaaaaaaatattcctttataaatgtatatacccatctcttcaccagcacaaccttaccttccattttctgcaacttctccaaatctcatactttccagaaaatcattttcccaagaaaaataaaactttcccctttgttcttctccccccaacaggtcaggctagtattttgttgttccccaatcctttcaaacattatcttcttcataatattactttgcttcacttcttttggtccttaaaccaaatttttgttcttctttagtttccttttttcccgttttgatttgattctttttatgttatttagagagaaacaagattcacaaattctctgatcttctttaactctttaaaacttttctttttcacactctagatttaaattatccctatggttacaaaacaattttgttcttagtttataacttgtgtattaccatcctttcttgattaactcttgttaggagaatatgaatgtaagatcaataaagttcttcaactttataattccttctataagatgtgtgtcggcaacagaattaaattaaatctttatagtttaactttaatctcaaccataattcaaaagaatagaaaacatgaagaatcttgagtcttttcaagaaaatcttgattggttttttgttggattcttgtaattctgtactaatcaaattttgccctaaacgtttttggctttggagcagcaatcacggcgtaccaatcggagctaggaggagattcctctcccttgaggaaatctgggagaggaaagatcgaaatcaaacggatcgagaacacaacgaatcgtcaagtcactttttgcaaacgtagaaatggtttgctcaagaaagcttacgagctctctgttctttgtgatgctgaagtcgcactcatcgtcttctctagccgtggtcgtctctatgagtactctaacaacaggtttcttcttcttctcgtgctctgttcttactttattaataattaaattatttttaaagtccgatttagggttttatgtttatgttaaagcataaatcttttacgagggttttcgatcttctaagctagatttgattctcttcttcttgaatgctcttatgggtaggattatttttcacttttttccttcatattccacacacatatatatataaacacactaacattagtgggaatatttgtttgatatgtttattttatttacttcgggggtttttgtaacaattttgtagatctaatttcttgttcttcatgtgtatattaattttcccttaagacttaaataaaaagagagaatttgttatatatagatatatgaagtgagggaaatggtacaaagttaaaggagatctgagtgagagttagataataaatgaaaagaaataagaaaccatcagggttttttctaatgtggagttttagattcagttttgtagaactaagattcactttgttgggtgttctttcttcactcatttctgttattataataataataaaatcttatatctttctattttccttactaacaagtacttgaagatttagatatatttatagatctggtgttgtaataggtaaaaacttgatttttatgactataaaagtaagttttgggaaacaaattggggagagagtaaggaaggactatgaggtcatatcttctgttttgtgatcatccatcctccattgttgttaatgtctgtgtctctctttttcttctcttctttctcttactttcctttcttatctctagctctctttctctctcatgaattatatcatatcatatatttgatacaaacacatgtgatggtaagtgagagtgaataaggtgaaactagctagatttttgagttttcatgaaattttaacttatatgagtgatagaaaataatggaacttatacgtacatgtaggacaatttagatggttatctaagtttttgtttttgttttctcttgagaatgttaaatgttagtgttatttttgtagttttggaaaattatatatgagctaagattagtttagaagtggtcaaaagaaacatagatttgaaatttcaactgaattttcaagatttcaaatagtcaatgaaacaaggaggtaattaagacaaattagcttatggggactcttttttgttattccttaaaattactctttttaaaattaaaaataactaatctcatttcgaactacattactcaaactagtaatctctaattcgacacgcaatttccaaatacttattagtagagagtcccacgtgattactttcttctccaccaaaacataaaacatgtcaagattaaatggtgtttgaaaattaaaagatcaattttcttaatcgtttacagttgtcaactctcatgtcctgaaatatataattctcatgtccaaaacaagaaaagctaacaacgacttcaaattaaatcagtcaatcaaaattagtcttcatttacctactaatttctttttatatatccgatgggtactctacgaaatcagagtttcgtttctttatttattttcttttataagatttttgaggttttttcagaggttggaattgagcgcaagattaggttttgggtctgtaagatttgttgtctttgttaaagaatctttgatcacgtcatcactcagatattatttctttttatttttcacttgtatttttactaatttattataaagttttgttagtttcagttcttgacttctgacaagaaggttttatgtcataatgaattaatttgtaacctatttataaattcaaaaatgtcatcatattactacttttgaccatttaatattagatttctcatttggtcaatacccaatgttcatattacatatatagagacaaaaattataaggatactaaattgttcatatttcttggaagtaaaaagattaatgatcactgaataaatagatttggcatagaagtatagcattggaattgcttcaacatctttggtgtagatagatttatgcaatttctctttctttttgaagtatcttttttttttctagagagagaataatgttagggatttttatcattttctctctcattatgggtactgagaggaaagtgagatttttagtacggatccaatagtttaagagtttggtctgccttctacgatccaaaaaaatctacggtcatgatctctccatcgagaaggttgagagttcagacatcaaagtctataatatgtcattgtaatacgtatttgtgtatatatatctatgtacaagtacatatacaggaaactcaagaaaaaagaataaatggtaaatttaattatattccaaataaggaaagtatggaacgttgtgatgttactcggacaagtcatttagttacatccatcacgtttaaatttaatccaatggttacaattttaatactatcaaatgtctattggatttatacccaatgtgttaatgggttgttgacacatgtcacatgtctgaaaccctagacatgttcagaccaatcatgtcactctaattttgccagcatggcagttggcagccaatcactagctcgataaatttaaggtttcagaggaattttaatttatttagggttcatattgtttcataaaatgattctttatttgttacaactttaaggaaatattttattaactatttaattgttcccttttcttatattacttttgttttttcttcacatcatgtgtcacattaagttgcatttcttctgactcaaaagaaccgatgtttgcttttaaggtttcgtattagaatcacttaactgtgcaagtggtcgatttgaccctatcaattttattttttattacttatcaaaatgcagatttaagcgtagattaagtttagaaaataggtagttaataggtctaattaacttattaattcctttaaaaaaattattgcagtgtaaaagggactattgagaggtacaagaaggcaatatcggacaattctaacaccggatcggtggcagaaattaatgcacaggtaagtggtaacttaatattacacgaatgattttaattaatatatgatgacatgacaacattgttcattcttttactctttttttttttgttgttgttgtagtattatcaacaagaatc

SEQ ID NO: 8 - DNA, 666 bp, tomato
cctataccccttggataagggtagctctatctatatagtcaattattgtcttctgtctgttggtgtcgacatactcgactcagaaggtattgaggaatgatcgattctgggtcatttgtgtggttaatcaccctccaaatcaactaagtcatcctgaaggacaatatcctattttttctctcgtaggtttatcatttaaattactatcgcgtgataattttgtaacgtagaaaaataataccattaatccaaacgttatattcattaaaataattatgatacatttaaaaatatttcgtgacctctcaattattgcaaattctaagccatcccaagttttgaggctaattttttttactatactatttttacaaccacaaaaacataaaaaataaaaaataaaaaaaataaaccgagtcaattgctacaatcacttcattattaattttaattaatattatgtggttatatatgaaactgttagagaaataatagctccaccatatttttttctcaatttattttcactataaaaaggctatttcattataatcaaaacaagacacacacaaagagaaggagcaataaaataaaagtaaacaacaatttgtgtgtttaaaaaaaaaaaaaaagtacacacaccaaaaaaaaaaattccaatttaaa

SEQ ID NO: 9 - DNA, 76 bp, Zygosaccharomyces rouxii
ccgcgtggaccactttcctgaccctagtggcagcccagggtggatctccctaggactcgcagttgagctcagattt

SEQ ID NO: 10 - DNA, 1473 bp, Zygosaccharomyces rouxii
tcaggaggtgtcgatggatcgtccgacgacaatgctaatctttcggcgcttcacatgtgccgctgttaacgcgttataaagtgcagttctcccattatccggaaaatctgggacgcctcgggaacttcttgcttgtaacttatactctttaccttcgttattcttgaacctggcatagctgctcaaaaaaaccaatacatccattggtattatttctgcattctttccataccgagccatcaatgtctcgatatctgatatcataggaatattcttgtcttgttcacaagggttgctatttggatcaaccaactcacacgccctttcggctgtgatattataaaaccccgatagaaacgcaaataaataagaaggcggactcttttctatcgtgtgcatgtatcgagccttagccacacggctaacaccttcttcccgcgcggcagaccaattaccatagagcgtggcttctttatccatctcattatttgaaagatatgaagctgtcacatgcctgcctaaatgcgctttcggtccattgggtattttaaagatgctctcatccgattgtttagagataaacccgtcatatgagcctaacagactgttcctgagaagctggtaatcatagcgtgcatcctcatcagtagttcgagttttgggaataggatctgtccattggaggtatgaatctagtgccaacagcgggtcgcatcgtcctttacaagggaagaaatagacaaaacgagtaccagtcttagtctctgggacaaaagcacgtagcatgcggccaaggtgcttgtctggaataacttcaaatgtcttgatatcggtattctttaagtcgtctgctctacaacaattcataaaagtcgcctgaagcaaaagattgtacgcggcttttgttgtaggccgagtagttctagcttctattaagtccatagtcttcccgacaaacccccaaatagtttcctgtgtttcagcgattttagtgatttcatcattaatcttgttacccaaatctcttttccgacctgcttcctttctcatatgtacagcagataaatgactcattacatcatcaggcttttcgtgtacccccacaacaaatctatagggcgatacaacatcttcgagtccttttattagcttacttgggtctttcaaatggtattcaaaggataccgttttagtggaagaatcgtattgcaatgtttttgaaatagacctctggtatttcaagaaagtggagcgcttgacaggaacgtcctttcttttctgactcgccaaattggccatcaaaattatcatcgtcagatgggaggctagtttttccttaggtaaagggttctcattctccaaaatgtcttttattttagagatctggtgtaagggaaggatttgactaagctcgctgaagtctgacatttgtctattaattgttgaaatttcagtatccttggtcaattgcat

SEQ ID NO: 11 - DNA, 426 bp, Tobacco vein mottling virus
cggaaggatggtaattataggtacccatgctgctgcgtcactctcgaagatggtagtccaatgtactcagagcttaaaatgccaacgaaaaatcatctagtaattggcaattcaggggatccgaaatacttggatctaccaggtgaaattagcaatcttatgtacatagcaaaggaaggatattgttatatcaacatatttcttgcaatgcttgttaatgttgatgaagctaacgccaaggactttactaagagagtgagggacgagtcagtacaaaagcttggaaagtggccaagtttaatagatgtcgcaactgaatgtgccttactatctacatattatcctgcggcggctagtgcagaactacccaggcttctagtagatcatgctcaaaagacaattcacgttgtggattcttatgggtcg